(19) United States
(12) Patent Application Publication
(10) Pub. No.: US 2003/0053972 A1
(43) Pub. Date: Mar. 20, 2003

(54) Colored shampoo lather and containers therefor

(76) Inventor: Travis Newell, Clovis, CA (US)

Correspondence Address: MARK D MILLER, KIMBLE, MACMICHAEL & UPTON, 5260 NORTH PALM AVENUE, SUITE 221, FRESNO, CA, 93704, US

(21) Appl. No.: 09/957,149

(22) Filed: Sep. 19, 2001

(51) Int. Cl. A61K 7/06

(52) U.S. Cl. 424/70.1

(57) ABSTRACT

The present invention provides a non-toxic shampoo concentrate that generates colored lather when it is mixed with water. The colored lather produced by the shampoo concentrate washes out and does not stain or color the hair it is applied to. The shampoo concentrate is provided in attractive colorful containers having a color scheme selected to correspond approximately with the color of the lather produced by the shampoo, and/or the shampoo concentrate itself. It is preferred that the concentrate be dispensed in bottles resembling crayons. Several small crayon-shaped bottles may be provided in a single package resembling an ordinary crayon box in both size and shape, with each of the small bottles sized such that it contains enough of the subject shampoo concentrate for approximately one application, enabling the user to select a different color of lather for each shampoo application.

### BACKGROUND OF THE INVENTION

1. Field of the Invention

The present invention relates generally to hair shampoo, and more particularly to shampoo for children which forms bright colorful (non-toxic) lather when mixed with water making shampoo time a fun time. The colored lather washes out and does not change the color of the hair, and may be provided in single-use crayon-shaped containers.

2. Description of the Prior Art

Different hair shampoo products are well known in the art. It is important that all shampoo products provide a satisfactory level of cleansing for proper hygiene. Beyond this basic requirement, numerous shampoo products have been developed over time which address related hair and scalp issues such as conditioning and control of dandruff. Shampoo products have also been developed to have aesthetically pleasing qualities such as colored liquid, creamy feel, perfumed smell and thick lather.

It is a well known phenomena that many people, particularly young children, shy away from regular or proper use of shampoo thereby risking maladies associated with improper hygiene. It is therefore desirable to find ways to encourage more and better use of shampoo among young children and others.

U.S. Pat. Nos. 4,450,091 and 4,544,495 are directed to shampoo formulations that produce high foam. U.S. Pat. No. 5,723,111 is directed to a composition for boosting shampoo foam, which may include adding perfumes or colorants to the composition itself. U.S. Pat. No. 4,741,854 is directed to a composition for reducing the color in transparent soap bars. While each of these inventions addresses a characteristic of shampoo use, none of them are directed towards changing the color of the foam or lather created by the shampoo formulation when it is mixed with water and applied to the user's hair. In these inventions, the lather or foam so produced is white, regardless of the color or consistency of the liquid shampoo formulation prior to mixing with water.

There are well known children's games that include adding food coloring to such things as shaving cream or detergent suds to create colored foam. U.S. Pat. No. 4,597,997 describes water based foam paints for watercolor artists. However, none of these uses relates to shampoo, and in all of these cases a pigment or dye is added to the foam to give it color, instead of being generated by the mixture of the underlying soap product with water. Moreover, the added pigment or dye will stain whatever it comes into contact with. It is not acceptable for shampoo to stain the hair it is to be applied to.

It is therefore desirable to provide shampoo which creates colored lather or foam when mixed with water that will not stain or discolor the hair it is applied to.

### SUMMARY OF THE INVENTION

The present invention provides a shampoo concentrate that generates colored lather when it is mixed with water. The colored lather produced by the shampoo concentrate washes out and does not stain or color the hair it is applied to. It should be non-toxic, and may be provided in tear-free formulations. The underlying shampoo concentrate may be of any color, or have no color so long as when it is mixed with water it produces colored foam, suds or lather. A wide variety of different colors may be produced by the shampoo, and may be as simple as the primary colors (red, yellow and blue), or they may be as varied as a rainbow, with all shades of color in between, including black and white. The shampoo concentrate may also be perfumed, if desired.

The shampoo concentrate of the present invention that creates colored lather may be provided in attractive colorful containers or bottles, the color of which may be selected to correspond approximately with the color of the lather produced by the shampoo. By way of example and for illustrative purposes only, and without limiting the scope of the appended claims, the liquid shampoo concentrate may be dispensed in an orange container or bottle. When the shampoo concentrate from the orange container is mixed with water, it forms lather having an orange color. The liquid shampoo concentrate may also be orange in color, but not necessarily. If the liquid shampoo concentrate is not orange in color, it may serve to have a “surprise” effect on small children, enhancing the colorful experience of hair washing.

The present invention includes a wide variety of different styles, shapes and sizes of bottles for holding the liquid shampoo concentrate. The bottles may be of rigid or deformable construction, and may be made of any suitable material such as plastic or glass. The bottles may be transparent, translucent or opaque, and may be in any desired color. One such bottle is in the form of a crayon, having a generally cylindrical body, a flat end and a pointed opposite end. The crayon-shaped bottle may be colored, as above, to correspond approximately to the color of the lather produced by the liquid shampoo concentrate in the bottle. Or the bottle may be transparent, allowing the color of the shampoo concentrate (which may or may not be orange) to show through.

A variety of different colored crayon-shaped bottles may be provided, in large or small sizes, each containing a different shampoo formula that produces a different colored lather corresponding generally to the color of the bottle and/or its labeling and/or its cap. A large bottle (e.g. 40 oz.) that is capable of standing on its own may be provided for dispensing the shampoo. When the shampoo is used up, the attractive large container may be used on a shelf or desk as a pencil holder, bank or other storage space. Several small crayon-shaped bottles may be provided in a single package resembling an ordinary crayon package in both size and shape. Each of the small bottles may be sized such that it contains enough of the shampoo concentrate of the present invention for approximately one application, enabling the user to select a different color of lather for each shampoo application.

The crayon shaped bottles may be in the form of a single deformable unit having a removable end cap allowing the shampoo inside to be dispensed through an opening in the pointed end of the bottle. After the cap is removed, the bottle may be squeezed to force the shampoo through the opening. Alternatively, the end cap may form the point on the crayon shaped bottle, such that when it is removed, it exposes a large opening at the end of the cylindrical bottle.

The present invention may also be used with shampoo for use on animals such as house pets including dogs, cats and other hairy or furry animals.

It is therefore a primary object of the present invention to provide a shampoo that produces colored lather when mixed with water, the colored lather having no effect on the color of the hair to which it is applied.

It is also a primary object of the present invention to provide a convenient and attractive container in the shape of a crayon for dispensing shampoo that produces colored lather when mixed with water, the colored lather having no effect on the color of the hair to which it is applied.

It is another object of the present invention to provide a shampoo product that encourages young children and others to shampoo their hair.

It is also an object of the present invention to provide a small crayon shaped container for holding enough shampoo for a single application, the shampoo producing colored lather when mixed with water, the colored lather having no effect on the color of the hair to which it is applied.

It is another object of the present invention to provide an assortment pack of small crayon shaped containers for holding different formulations of shampoo each of which produces a different color of lather upon mixing with water, the color of the container or its labeling generally corresponding to the color of the lather produced, the colored lather having no effect on the color of the hair to which it is applied.

Additional objects of the invention will be apparent from the detailed descriptions and the claims herein.

### BRIEF DESCRIPTION OF THE DRAWINGS

FIG. 1 is a side view of a container for the shampoo of the present invention.

FIG. 1A is a side view of the cap removed from the container of FIG. 1.

FIG. 2 is a side view of another container with the cap removed.

FIG. 3 is a top view of the container of FIG. 2 with the cap removed.

FIG. 4 is a side view of the cap of the container of FIG. 2.

FIG. 5 is a side view of an alternative container with the cap removed.

FIG. 6 is a bottom view of the container of FIG. 5.

FIG. 7 is a side view of the cap of the container of FIG. 5.

FIG. 8 is a view showing a child lathering up using the shampoo of the present invention.

### DETAILED DESCRIPTION OF THE PREFERRED EMBODIMENTS

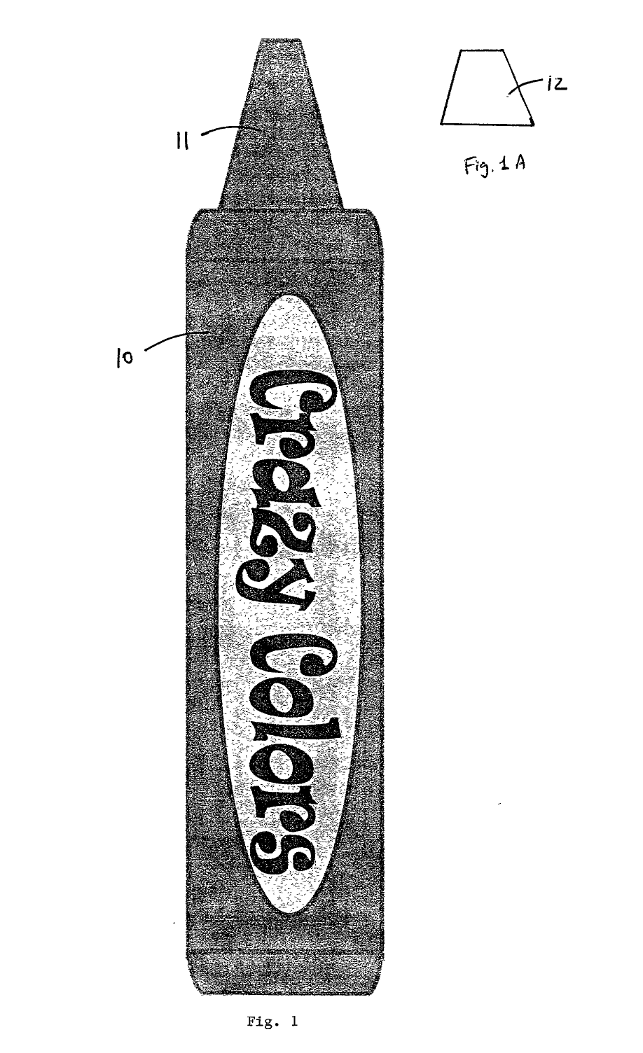

Referring to the drawings wherein like reference characters designate like or corresponding parts throughout the several views, and referring particularly to Figs. 1-1A, it is seen that the preferred containers for the invention are in the form of crayons. The container of FIG. 1 has an integrated a pointed end 11 attached to a deformable main body 10. Removal of cap 12 exposes a small opening 14 at the tip of point 11 through which liquid shampoo concentrate may be squeezed for application.

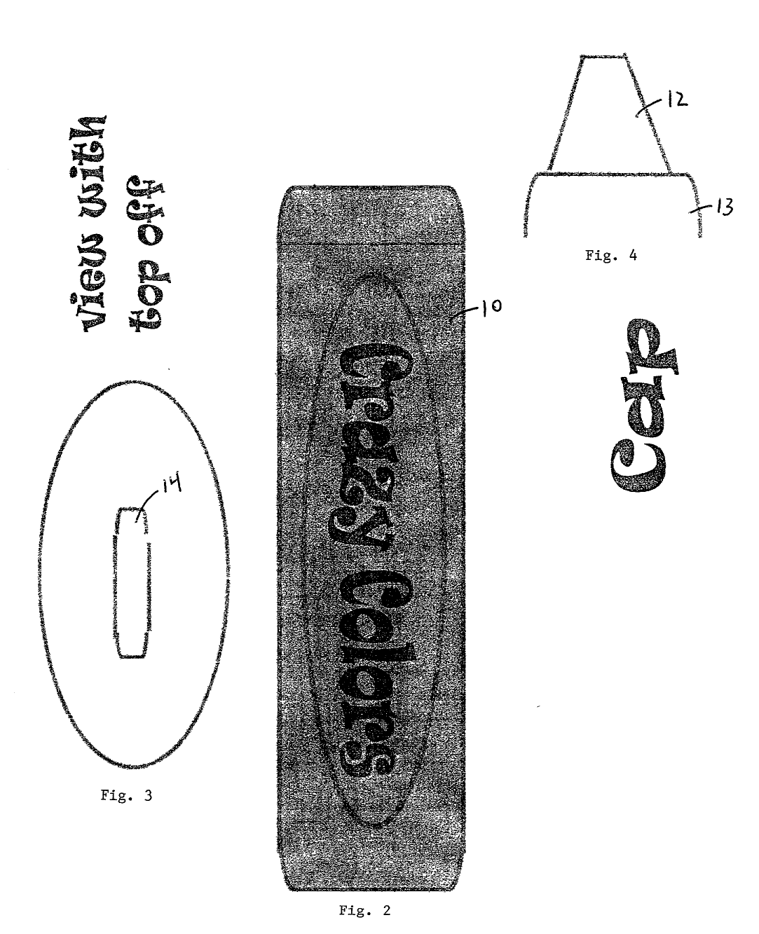

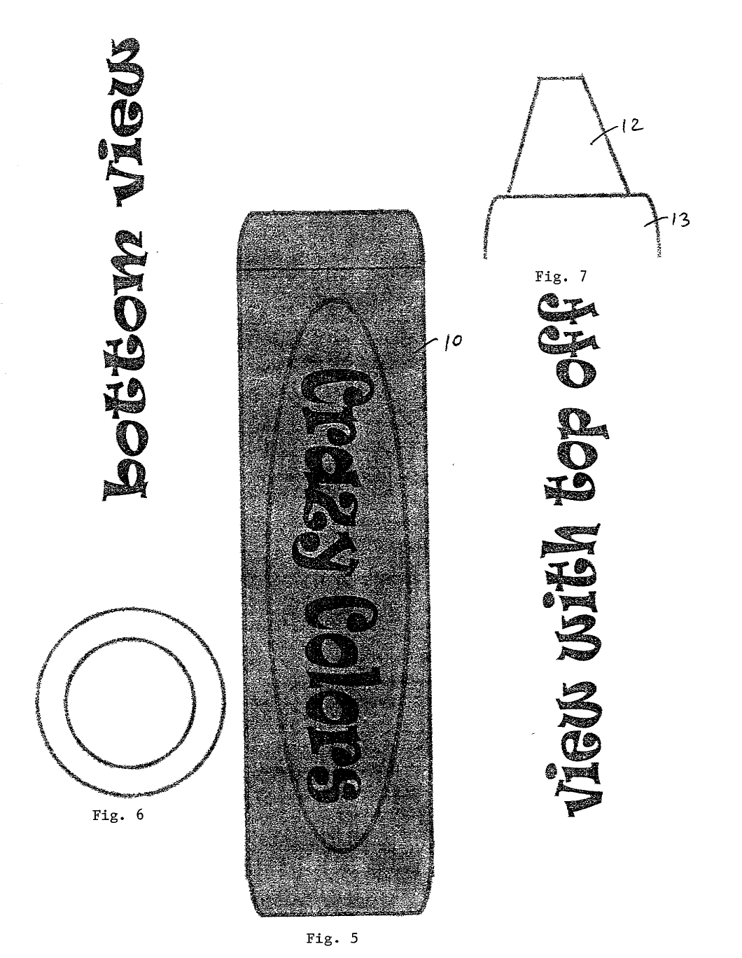

FIGS. 2-4 illustrate another embodiment of the crayon-shaped container in which body 10 has an oval cross section, but no point 11. Instead, the point is provided on cap 12 which also includes an annular shoulder 13 which fits over the end of body 10. Removal of cap 12 and shoulder 13 exposes an opening 14 at the top of body 10 through which liquid shampoo concentrate may be dispensed. In this embodiment, body member 10 may or may not be deformable. FIGS. 5-7 illustrate another embodiment having a body member 10 with a round cross section and no point, the point being provided in cap 12 with annular shoulder 13. A large opening 14 is exposed in this embodiment upon removal of cap 12, allowing liquid shampoo concentrate to be poured out of the container. This embodiment facilitates use of a rigid non-deformable body member 10, although a deformable body member may also be used.

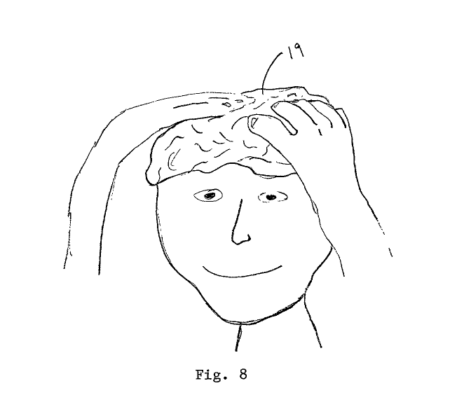

FIG. 8 shows a child lathering up shampoo in the hair. When water is added, the foam or lather 19 that is produced has a unique color, according to the present invention. The coloring of the shampoo foam does not affect the color of the child's hair.

It is preferred that the color of the liquid shampoo concentrate be approximately the same as the color of the lather produced by the concentrate. Thus, green shampoo concentrate should produce green lather foam when mixed with water. Using the same color for the concentrate and the lather facilitates the use of transparent bottles to be used to hold the shampoo.

It is also preferred that the crayon shaped body members for holding the liquid shampoo concentrate be deformable, allowing the shampoo to be squeezed out, and providing a play toy after the shampoo is used up.

It is to be appreciated that the colored lather of the present invention need not be limited to use with only shampoo or hair care products, but that it may be used with any solid or liquid soap or cleaning material, including without limitation hand soap (bar soap or liquid soap), body and bath soap, toothpaste, and the like.

It is to be understood that variations and modifications of the present invention may be made without departing from the scope thereof. It is also to be understood that the present invention is not to be limited by the specific embodiments disclosed herein, but only in accordance with the appended claims when read in light of the foregoing specification.

What is claimed is:

1. Hair shampoo comprising a formula that forms colored lather upon mixing with water.

2. The shampoo of claim 1 wherein said colored lather does not change the color of the hair to which it is applied.

3. A container for hair shampoo comprising a bottle in the shape of a crayon for holding shampoo that forms colored lather upon mixing with water.

4. The container of claim 3 wherein said colored lather does not change the color of the hair to which it is applied.

5. The container of claim 4 wherein the color of the bottle is approximately the same as the color of the lather produced by the shampoo contained therein.

6. The container of claim 5 wherein said bottles has the approximate size of a crayon.

7. The container of claim 5 wherein said bottle is deformable.

8. An assortment of containers for hair shampoo comprising a plurality of bottles in the shape of crayons for holding shampoo that forms colored lather upon mixing with water wherein said colored lather does not change the color of the hair to which it is applied.

9. The assortment of claim 8 wherein each of said bottles has the approximate size of a crayon, and said plurality of bottles is provided in a crayon box.

10. The assortment of claim 9 wherein the color of each container is approximately the same as the color of the lather produced by the shampoo contained therein.

11. The assortment of claim 10 wherein each of said bottles is deformable.

12. The combination of a container for holding hair shampoo and shampoo comprising a bottle in the shape of a crayon and shampoo that forms colored lather upon mixing with water.

13. The combination of claim 12 wherein said colored lather does not change the color of the hair to which it is applied.

14. The combination of claim 13 wherein the color of the bottle is approximately the same as the color of the lather produced by the shampoo contained therein.

15. The combination of claim 14 wherein said bottle has the approximate size of a crayon.

16. The combination of claim 15 wherein said bottle is deformable.
